(19) United States
(12) Patent Application Publication
(10) Pub. No.: US 2011/0319461 A1
(43) Pub. Date: Dec. 29, 2011

(54) NOVEL SALTS, POLYMORPHS, AND SYNTHETIC PROCESSES REGARDING IMIDAZOLE DERIVATIVE

(75) Inventors: John Joseph Partridge, Chapel Hill, NC (US); John Frederick Reinhard, JR., Billerica, MA (US); Robert P. Rodebaugh, Holly Springs, NC (US); Jeff M. Servesko, Durham, NC (US)

(73) Assignee: Ore Pharmaceuticals Inc., Cambridge, MA (US)

(21) Appl. No.: 13/005,173

(22) Filed: Jan. 12, 2011

Related U.S. Application Data
(60) Provisional application No. 61/300,709, filed on Feb. 2, 2010.
(60) Provisional application No. 61/294,594, filed on Jan. 13, 2010.

(51) Int. Cl. A61K 31/4164; A61P 29/00; A61P 1/00; C07D 233/64

(52) U.S. Cl. 514/400; 548/339.1

(57) ABSTRACT

The present invention relates to a process for producing 2-[1-(S)-carboxy-2(S)-[3-(3,5-dichloro-benzyl)-3H-imidazol-4-yl]-ethylamino]-4-methyl-pentanoic acid, as well as novel salts, including hydrates and solvates thereof, and novel crystalline and amorphous forms thereof.

### CROSS-REFERENCE TO RELATED APPLICATIONS

The present invention claims benefit to U.S. Provisional Application No. 61/294,594, filed Jan. 13, 2010 and U.S. Provisional Application No. 61/300,709, filed Feb. 2, 2010, each of which is incorporated herein by reference in its entirety.

### FIELD OF THE INVENTION

The present invention relates to a process for producing 2-[1-(S)-carboxy-2(S)-[3-(3,5-dichloro-benzyl)-3H-imidazol-4-yl]-ethylamino]-4-methyl-pentanoic acid, as well as novel salts, including hydrates and solvates thereof, and novel crystalline forms, amorphous forms, and mixtures thereof.

### BACKGROUND OF THE INVENTION

Ulcerative colitis (UC) is a chronic inflammatory disease primarily affecting the colonic mucosa. The extent and severity of colonic involvement is variable. In the most limited form it may be restricted to the distal rectum, while in its most extended form the entire colon is involved.

The incidence of UC has been increasing in the western world since the early 1950's, largely attributed to better and more uniform diagnostic criteria. More recent trends indicate a change in the epidemiology of UC with previously low incidence areas now reporting a progressive rise in incidence. The western literature typically reports an incidence of approximately 6-8 cases per 100,000 population and an estimated prevalence of approximately 70-150 cases per 100,000 population.

The typical age of initial presentation of UC is between the 15 to 35 years with no clear gender distribution and it appears to run in families. The exact cause of ulcerative colitis remains unknown. However, genetic susceptibility, abnormal immune response, presence of luminal microorganisms and other environmental, factors including diet and ingested toxins have been implicated in the etiology. One of the more firmly established risk factors is a strong correlation with family history. Approximately 10 percent of subjects with ulcerative colitis have a first-degree relative with the illness. Whole genome scans have found susceptibility genes for UC on chromosome 3, 7 and 12 although these loci have not been uniformly confirmed.

Among the possible environmental factors, no specific foods have been identified as a cause of ulcerative colitis. However, a significant number of subjects diagnosed with UC report intolerance to cows' milk and report that dairy products may aggravate symptoms. Cigarette smoking actually reduces the risk and cessation of smoking seems to increase the risk of developing UC.

The most common symptoms of UC are pain in the abdomen and bloody diarrhea. Other symptoms may include anemia, severe tiredness, weight loss, loss of appetite, bleeding from the rectum, sores on the skin and joint pain. There are reports that demonstrate that UC could progress to colonic cancer over a period of 8-10 years. Other complications of UC include toxic megacolon, perforation, iron deficiency anemia and primary sclerosing cholangitis.

Angiotensin-converting enzyme 2 (ACE2) is a recently described angiotensin-converting enzyme (ACE) homologue. ACE2 is a zinc metallopeptidase that catalyzes the conversion of angiotensin I (Ang I) and angiotensin II (Ang II) to angiotensin (1-9) and angiotensin (1-7), respectively. Other substrates of this enzyme include ghrelin, apelin, dynorphin, bradykinin, and neurotensin. Attempts to elucidate the biological role of ACE2 have included knock-out animal models, each being viable with phenotypes highly dependent on background strain. Among its purported functions, ACE2 is believed to be a component of the renin-angiotensin system (RAS), the dysregulation of which has been implicated in a number of disease states.

ACE2 expression has also been found in epithelial and submucosal cells throughout the gastrointestinal tract, with significant expression in the ileum and colon. These findings are consistent with colon expression of other RAS components such as AT1 and AT2 receptors, renin, and ACE. While the function of ACE2 in the gastrointestinal tract is unknown, recent expression profiling studies revealed ACE2 mRNA overexpression in the stomach and colon of patients with chronic gastritis and IBD, respectively.

Compound (I), a potent and selective ACE2 inhibitor, exhibits anti-inflammatory effects in the upper gastrointestinal tract of the mouse and protects against NSAID-induced gastric damage in rats. It is therefore likely that inhibition of ACE2 with Compound (I) may be of utility in the treatment of inflammatory diseases of the gastrointestinal tract including inflammatory bowel disease (IBD).

2-[1-(S)-carboxy-2(S)-[3-(3,5-dichloro-benzyl)-3H-imidazol-4-yl]-ethylamino]-4-methyl-pentanoic acid, herein also referred to as Compound (I), is described in U.S. Pat. No. 6,632,830, U.S. Pat. No. 7,045,532, and WO 00/066104, each herein incorporated by reference in their entirety. Additionally, Compound (I) is discussed in “Substrate-Based Design of the First Class of Angiotensin-Converting Enzyme-Related Carboxypeptidase (ACE2) Inhibitors”, Dales, N. A., et al., J. Amer. Chem. Soc., 2002, 124, 11852-11853.

### 2-[1-(S)-carboxy-2(S)-[3-(3,5-dichloro-benzyl)-3H-imidazol-4-yl]-ethylamino]-4-methylpentanoic acid (Compound I)

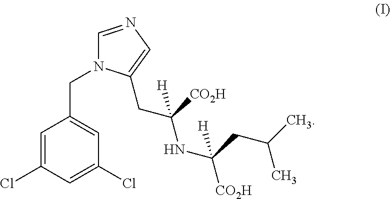

The commercial development of a drug candidate involves many steps, including the development of a cost effective synthetic method that is adaptable to a large scale manufacturing process. Commercial development also involves research regarding salt forms of the drug substance that exhibit suitable purity, chemical stability, pharmaceutical properties, and characteristics that facilitate convenient handling and processing. Furthermore, compositions containing the drug substance should have adequate shelf life. That is, they should not exhibit significant changes in physicochemical characteristics such as, but not limited to, chemical composition, water content, density, hygroscopicity, stability, and solubility upon storage over an appreciable period of time. Additionally, reproducible and constant plasma concentration profiles of drug upon administration to a patient are also important factors. Solid salt forms are generally preferred for oral formulations due to their tendency to exhibit these properties in a preferential way; and in the case of basic drugs, acid addition salts are often preferred salt. However, different salt forms vary greatly in their ability to impart these properties and such properties cannot be predicted with reasonable accuracy. For example, some salts are solids at ambient temperatures, while other salts are liquids, viscous oils, or gums at ambient temperatures. Furthermore, some salt forms are stable to heat and light under extreme conditions and others readily decompose under much milder conditions. Salts also vary greatly in their hygroscopicity, the less hygroscopic being more advantageous. Thus, the development of a suitable acid addition salt form of a basic drug for use in a pharmaceutical composition is a highly unpredictable process.

As noted in Dales et al., as cited above, and appreciated by those skilled in the art, Compound (I) contains two stereocenters thereby providing 4 stereoisomers. The R,R isomer is inactive (>50 μM). Dales et al. report the remaining isomers to be equipotent, with the S,S isomer providing selectivity against ACE and CPDA (IC₅₀'s>50 μM).

Because of the advantageous pharmacological properties of a single enantiomer over its racemate, there is a need for a stereospecific synthesis, preferably a process suitable for large-scale production. Furthermore, there is a need for salt forms that display improved properties, such as for example purity, stability, solubility, and bioavailability. Preferential characteristics of these novel salt forms include those that would increase the ease or efficiency of manufacture of the active ingredient and its pharmaceutical composition into a commercial drug product and improved stability of the drug over a prolonged period of time.

### SUMMARY OF THE INVENTION

Neither the dicarboxylic acid (also referred to as a “zwitterion”) nor the disodium salt anhydrate is ideal for pharmaceutical formulation. The dicarboxylic acid has limited water solubility and the disodium salt anhyrate is unstable, thermally sensitive, and subject to oxidation at ambient temperatures. The present invention provides crystalline forms with improved physical properties, improved chemical stability, and fewer impurities for the preparation of solid pharmaceutical dosage forms.

One aspect of the present invention is a process for preparing Compound (I)

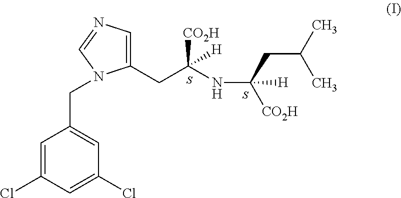

or a pharmaceutically acceptable salt, or hydrate, or solvate thereof, comprising an enantioselective reductive amination. In one embodiment, the process further comprises the use of pivaloyloxyborohydride or a salt thereof. In a further embodiment, the pivolyl borohydride is used in a 15:1 ratio. In a further embodiment, the process further comprises a subsequent acetone wash. One aspect of the present invention is the product prepared by such process.

Another aspect of the present invention is a process for preparing Compound (I),

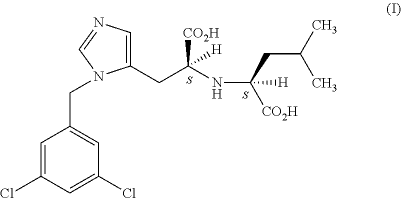

or a salt, hydrate, solvate, or polymorph thereof, comprising the steps of:

(a) acylating a compound of formula (II)

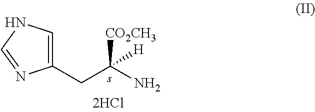

or a salt thereof, with di-tert-butyl dicarbonate—(Boc)₂O to form the compound of formula (III)

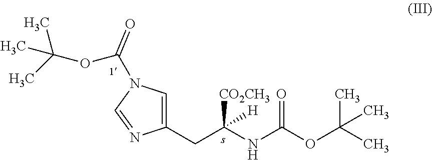

(b) reacting 3,5-dichlorobenzyl alcohol of formula (IV) with triflic anhydride and diisopropylethylamine base

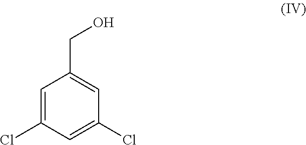

to form labile 3,5-dichlorobenzyl triflate of formula (V)

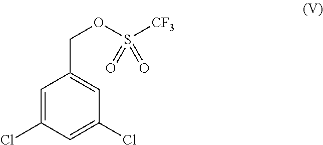

(c) coupling of the compound of formula (III) with 3,5-dichlorobenzyl triflate of formula (V) to produce intermediate compound of formula (VI)

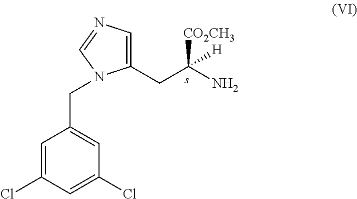

(d) coupling of the compound of formula (VI) with 4-methyl-2-oxovaleric acid to form Schiff base mixture of formula (VII)

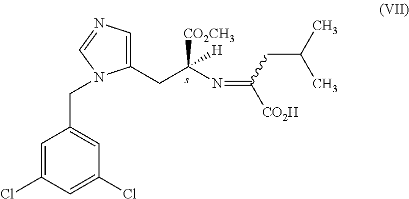

(e) reductive amination of the Schiff base mixture of formula (VII) in the presence of sodium tri(pivaloyloxy)borohydride to form a compound of formula (VIII) as a mixture of diastereoisomers

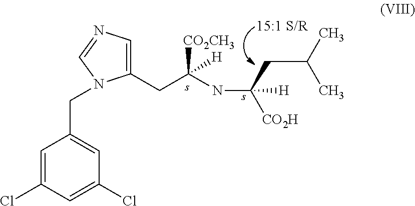

(f) saponification of the compound of formula (VIII) with aqueous sodium hydroxide, followed by acidification with aqueous hydrochloric acid and isolating the resulting solid product.

In one embodiment, the salt of compound (I) is a monosodium salt, a disodium salt, a monopotassium salt, a dipotassium salt, a monoammonium salt or a diammonium salt, or a combination thereof containing sodium, potassium or ammonium counterions, or a hydrate of solvate thereof.

In one embodiment, the salt of compound (I) is a disodium salt of formula (IX)

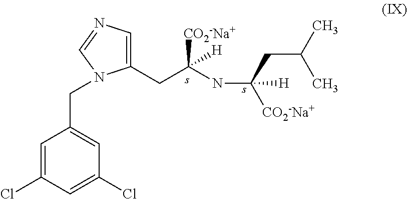

or a hydrate or solvate thereof.

In one embodiment, the salt of compound (I) is a disodium tetrahydrate salt of formula (X)

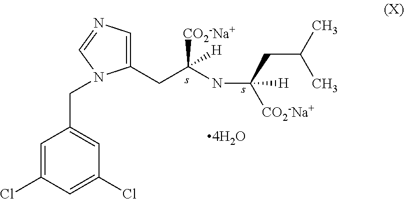

In one embodiment, the salt of compound (I) is a monohydrochloride salt, a dihydrochloride salt, a bisulfate salt, a sulfate salt, or a phosphate salt, or a hydrate or solvate thereof.

In one embodiment, the salt of compound (I) is a monohydrochloride salt of formula (XI)

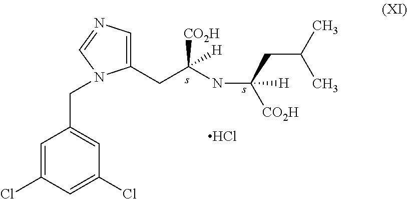

or a hydrate or solvate thereof.

In one embodiment, the salt of compound (I) is a dihydrochloride salt of formula (XII)

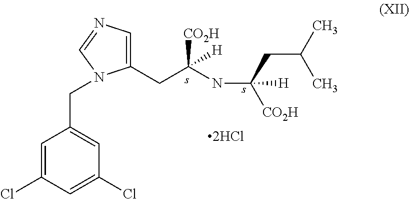

or a hydrate or solvate thereof.

In one embodiment, the compound of formula (X) was crystallized from an aqueous sodium hydroxide and acetone mixture.

Another aspect of the present invention includes a process for preparing

Compound (I)

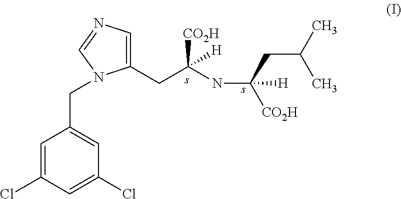

or a pharmaceutically acceptable salt, or hydrate, or solvate thereof, comprising use of an intermediate of formula (VII)

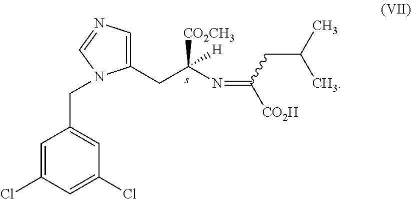

Another aspect of the present invention includes a compound of Formula (IX) or a solvate or hydrate thereof

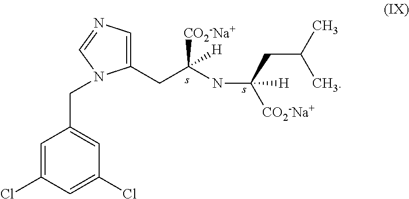

In one embodiment, the compound is amorphous.

Another aspect of the present invention includes a compound of formula (X) or a solvate of hydrate thereof

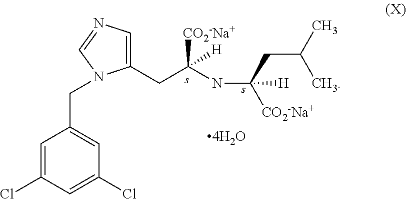

In one embodiment, the compound is amorphous. In another embodiment, the compound is crystalline. In another embodiment, the compound is crystalline and is substantially free of amorphous.

Another aspect of the present invention is a compound of formula (XI) or a solvate of hydrate thereof

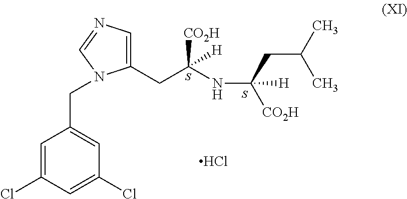

Another aspect of the present invention is a compound of formula (XII) or a solvate of hydrate thereof

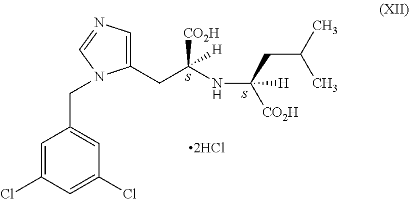

In one embodiment, the compound is a hydrate comprising up to about 10 mole percent water.

Another aspect of the present invention includes a polymorphic form of a compound of Formula (X)

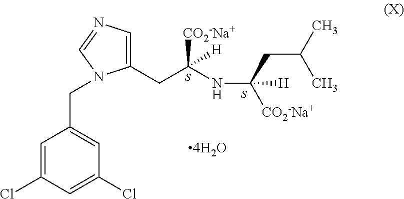

characterized by a powder x-ray diffraction pattern comprising at least one of the following peaks:

2θ

4
16
16.8
19.8

Another aspect of the present invention includes a polymorphic form of a compound of Formula (X)

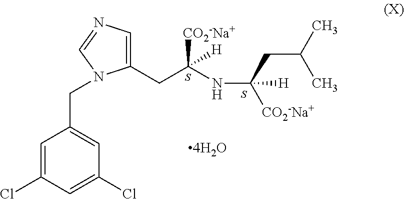

characterized by a powder x-ray diffraction pattern that substantially corresponds to that shown in FIG. 2.

Another aspect of the present invention includes a hydrated form of compound (IX)

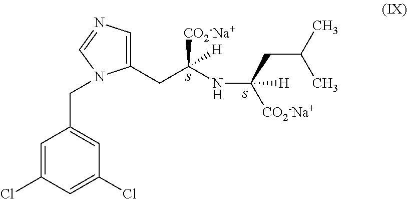

having a sharp endotherm from between about 87 to about 91° C.

Another aspect of the present invention includes a compound of formula (X)

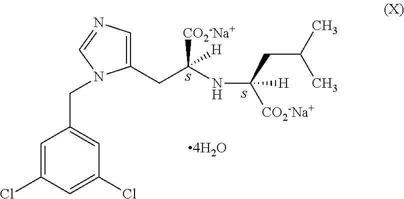

characterized by a differential scanning calorimetry thermogram that substantially corresponds to that shown in any one of FIG. 5, FIG. 6, or FIG. 7.

Another aspect of the present invention includes a pharmaceutical composition comprising a compound of the present invention and one or more pharmaceutically acceptable carriers, diluents, or excipients. In one embodiment, the pharmaceutical composition is an oral dosage form.

Another aspect of the present invention includes an oral dosage form comprising a compound of the present invention and one or more pharmaceutically acceptable carriers, diluents, or excipients.

Another aspect of the present invention includes a method of treating or preventing inflammatory disorders of the gastrointestinal tract comprising administering a compound of the present invention.

Another aspect of the present invention includes use of a compound of the present invention in the manufacture of medicament for the treatment or prevention of inflammatory disorders of the gastrointestinal tract.

Another aspect of the present invention includes a compound of the present invention for use in treating or preventing inflammatory disorders of the gastrointestinal tract.

Combinations of aspects and embodiments form further embodiments of the present invention.

### BRIEF DESCRIPTION OF THE FIGURES

FIG. 1 is an X-ray diffraction pattern of a crystalline form of Compound (I).

FIG. 2 is an X-ray diffraction pattern of a crystalline form of Compound (I).

FIG. 3 is an X-ray diffraction pattern of a crystalline form of Compound (I).

FIG. 4 is an X-ray diffraction pattern of a partially crystalline form of Compound (I).

FIG. 5 is a differential scanning calorimetry thermogram for a crystalline form of Compound (I).

FIG. 6 is a differential scanning calorimetry thermogram for a crystalline form of Compound (I).

FIG. 7 is a differential scanning calorimetry thermogram for a crystalline form of Compound (I).

FIG. 8 is a differential scanning calorimetry thermogram for a partially crystalline form of Compound (I).

### DETAILED DESCRIPTION OF THE INVENTION

The following definitions are meant to clarify, but not limit, the terms defined. If a particular term used herein is not specifically defined, such term should not be considered indefinite. Rather, terms are used within their accepted meanings.

As used herein, the term “compound” may be used to mean the free base form, or alternatively, a salt form of 2-[1-(S)-carboxy-2(S)-[3-(3,5-dichloro-benzyl)-3H-imidazol-4-yl]-ethylamino]-4-methylpentanoic acid (Compound I):

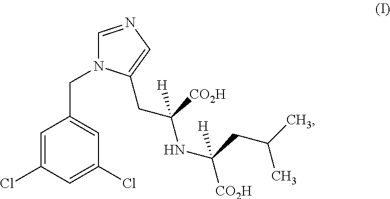

depending on the context, which will be readily apparent. Those skilled in the art will be able to distinguish the difference. The present invention includes any amorphous, partially crystalline or crystalline forms, including glasses, lyophilates, and mixtures thereof. The present invention also includes all salts, hydrates, solvates, and mixtures thereof.

Compound 1 exists in several specific crystalline polymorphs and may exist as a mixture of crystalline and amorphous material. These specific polymorphs may be the reason for certain desirable pharmaceutical properties, therefore, the identification of specific and preferred polymorphs is an aspect of the present invention.

Specific crystalline polymorphs have desirable properties for a pharmaceutical preparation. For example, Compound (I) appears very stable at ambient temperature and humidity with no apparent degradation over the course of 7 years. Compound (I) has excellent water solubility, allowing for both rapid dissolution of capsule or tablet formulations and the ability to provide intravenous formulations, if desired. Preferred forms of Compound (I) have relatively high density, thereby enabling capsule or tablet formulations. Preferred forms of Compound (I) are stable in gelatin capsules, allowing for their use in this particular formulation.

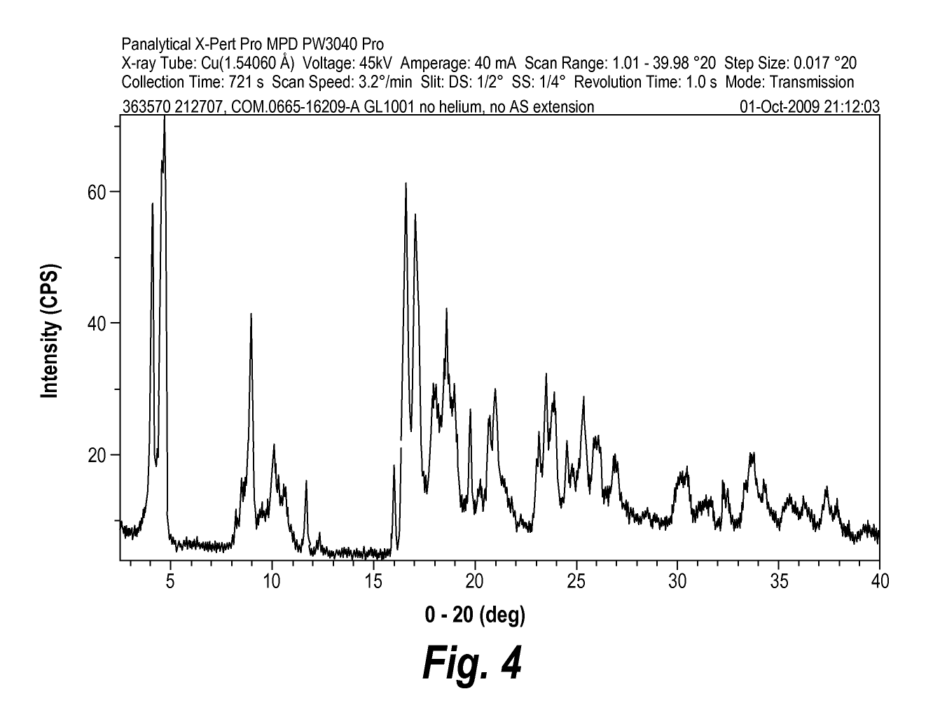

As depicted in FIG. 4, one form provides a mixture of crystal and amorphous material. As such, this material is incompatible with gelatin capsules, with capsule breakdown occurring in less than one month. This material, a disodium salt, was believed to be a tetrahydrate form; however, upon DSC analysis, the material was determined As will be noted, therefore, the disodium salt is best provided as a tetrahydrate form.

As will be appreciated by those skilled in the art, different naming conventions may name a compound differently. Such naming conventions should not be used to introduce ambiguity to this specification.

As used herein, the phrase “pharmaceutically acceptable” refers to carrier(s), diluent(s), excipient(s) or salt forms of the compound of Formula I that are compatible with the other ingredients of the composition and not deleterious to the recipient of the pharmaceutical composition.

As used herein, the phrase “pharmaceutical grade” refers to a compound or composition of a standard suitable for use as a medicine. With reference to the discussion herein, pharmaceutical grade compounds of the present invention, particularly salt forms thereof, display appropriate properties, including purity, stability, solubility, and bioavailability for use in a drug product. Preferential characteristics include those that would increase the ease or efficiency of manufacture of the active ingredient and its composition into a commercial drug product. Furthermore, pharmaceutical grade compounds of the present invention may be synthesized using a stereospecific synthesis that is scalable to a large-scale production, namely displaying adequate purity and yield.

As used herein, the term “pharmaceutical composition” refers to a compound of the present invention optionally admixed with one or more pharmaceutically acceptable carriers, diluents, or excipients. Pharmaceutical compositions preferably exhibit a degree of stability to environmental conditions so as to make them suitable for manufacturing and commercialization purposes.

As used herein, the terms “effective amount”, “therapeutic amount”, or “effective dose” refer to an amount of the compound of the present invention sufficient to elicit the desired pharmacological or therapeutic effects, thus resulting in effective prevention or treatment of a disorder. Prevention of the disorder may be manifested by delaying or preventing the progression of the disorder, as well as the onset of the symptoms associated with the disorder. Treatment of the disorder may be manifested by a decrease or elimination of symptoms, inhibition or reversal of the progression of the disorder, as well as any other contribution to the well being of the patient.

Another aspect of the invention involves an asymmetric synthesis that involves a combination of reductive amination with pivaloyl borohydride and subsequent acetone wash steps to provide the final S,S diastereomer in >99% yield. While the use of the pivaloyl borohydride has been described in prior syntheses, the high level of enantiomeric selectivity attained under the present conditions is unexpected, novel, and of practical use.

As will be discussed in more detail below, the effective dose can vary, depending upon factors such as the condition of the patient, the severity of the symptoms of the disorder, and the manner in which the pharmaceutical composition is administered. Thus, as used herein, the effective dose may be 1500 mg, in another embodiment 900 mg, in another embodiment 300 mg, or in another embodiment 100 mg. Doses up to about 2100 mg/day may be administered. These effective doses typically represent the amount administered as a single dose, or as one or more doses administered over a 24 h period.

As used herein, the phrase “substantially' or ‘sufficiently’ quality, purity or pure, includes greater than 20%, preferably greater than 30%, and more preferably greater than 40% (e.g. greater than any of 50, 60, 70, 80, or 90%) quality or purity.

The term “stability” as defined herein includes chemical stability and solid state stability, where the phrase “chemical stability” includes the potential to store salts of the invention in an isolated form, or in the form of a pharmaceutical composition in which it is provided in admixture with pharmaceutically acceptable carriers, diluents, excipients, or adjuvants, such as in an oral dosage form, such as a tablet, capsule, or the like, under normal storage conditions, with an insignificant degree of chemical degradation or decomposition, and the phrase “solid state stability”, includes the potential to store salts of the invention in an isolated solid form, or in the form of a solid pharmaceutical composition in which it is provided in admixture with pharmaceutically acceptable carriers, diluents, excipients, or adjuvants, such as in an oral dosage form, such as a tablet, capsule, or the like, under normal storage conditions, with an insignificant degree of solid state transformation, such as crystallization, recrystallization, solid state phase transition, hydration, dehydration, solvation, or desolvation.

Examples of “normal storage conditions” include one or more of temperatures of between −80° C. and 50° C., preferably between 0° C. and 40° C. and more preferably ambient temperatures, such as 15° C. to 30° C., pressures of between 0.1 and 2 bars, preferably at atmospheric pressure, relative humidity of between 5 and 95%, preferably 10 to 60%, and exposure to 460 lux or less of UV/visible light, for prolonged periods, such as greater than or equal to six months. Under such conditions, salts of the invention may be found to be less than 5%, more preferably less than 2%, and especially less than 1%, chemically degraded or decomposed, or solid state transformed, as appropriate. The skilled person will appreciate that the above-mentioned upper and lower limits for temperature, pressure, and relative humidity represent extremes of normal storage conditions, and that certain combinations of these extremes will not be experienced during normal storage (e.g. a temperature of 50° C. and a pressure of 0.1 bar).

### Compounds

One embodiment of the present invention relates to salt forms of 2-[1-(S)-carboxy-2(S)-[3-(3,5-dichloro-benzyl)-3H-imidazol-4-yl]-ethylamino]-4-methylpentanoic acid.

In one embodiment, the compound or a pharmaceutically acceptable salt thereof is substantially pure. In one embodiment, the compound or a pharmaceutically acceptable salt thereof is substantially free of alternative enantiomers, racemates, and mixtures, including 2-[1-(R)-carboxy-2(S)-[3-(3,5-dichloro-benzyl)-3H-imidazol-4-yl]-ethylamino]-4-methylpentanoic acid, 2-[1-(S)-carboxy-2(R)-[3-(3,5-dichloro-benzyl)-3H-imidazol-4-yl]-ethylamino]-4-methylpentanoic acid, 2-[1-(R)-carboxy-2(R)-[3-(3,5-dichloro-benzyl)-3H-imidazol-4-yl]-ethylamino]-4-methylpentanoic acid, and racemic 2-[1-carboxy-[3-(3,5-dichloro-benzyl)-3H-imidazol-4-yl]-ethylamino]-4-methylpentanoic acid.

In one embodiment, the compound of Formula I or a pharmaceutically acceptable salt thereof is present in an amount of about 75% by weight compared to alternative enantiomers and mixtures, preferably greater than 85% by weight, more preferably greater than 95% by weight, more preferably greater than 98% by weight, and most preferably 99% by weight or greater. One embodiment relates to 100% pure 2-[1-(S)-carboxy-2(S)-[3-(3,5-dichloro-benzyl)-3H-imidazol-4-yl]-ethylamino]-4-methylpentanoic acid.

### Methods of Treatment

As used herein, inhibition of enzyme activity is denoted by the term “inhibitor”.

As used herein, the terms “prevention” or “prophylaxis” include any degree of reducing the progression of or delaying the onset of a disease, disorder, or condition. The term includes providing protective effects against a particular disease, disorder, or condition as well as amelioration of the recurrence of the disease, disorder, or condition. Thus, in another aspect, the invention provides a method for treating a subject having or at risk of developing or experiencing a recurrence of an ACE mediated disorder. The compounds and pharmaceutical compositions of the invention may be used to achieve a beneficial therapeutic or prophylactic effect.

Angiotensin-converting enzyme 2 (ACE2) is an angiotensin-converting enzyme (ACE) homologue. ACE2 is a zinc metallopeptidase that catalyzes the conversion of angiotensin I (Ang I) and angiotensin II (Ang II) to angiotensin (1-9) and angiotensin (1-7), respectively. Other substrates of this enzyme include ghrelin, apelin, dynorphin, bradykinin, and neurotensin. Attempts to elucidate the biological role of ACE2 have included knock-out animal models, each being viable with phenotypes highly dependent on background strain. Among its purported functions, ACE2 is believed to be a component of the renin-angiotensin system (RAS), the dysregulation of which has been implicated in a number of disease states.

ACE2 expression has also been found in epithelial and submucosal cells throughout the gastrointestinal tract, with significant expression in the ileum and colon. These findings are consistent with colon expression of other RAS components such as AT1 and AT2 receptors, renin, and ACE. While the function of ACE2 in the gastrointestinal tract is unknown, recent expression profiling studies revealed ACE2 mRNA overexpression in the stomach and colon of patients with chronic gastritis and IBD, respectively.

Compound (I), a potent and selective ACE2 inhibitor, exhibits anti-inflammatory effects in the upper gastrointestinal tract of the mouse and protects against NSAID-induced gastric damage in rats. Inhibition of ACE2 with Compound (I) is believed useful in the treatment of inflammatory diseases of the gastrointestinal tract, including but not limited to inflammatory bowel disease (IBD).

### Combinations

The compound of the present invention may be used in the treatment of a variety of disorders and conditions and, as such, may be used in combination with a variety of other therapeutic agents useful in the treatment or prophylaxis of those disorders. Thus, one embodiment of the present invention relates to the administration of the compound of the present invention in combination with other therapeutic agents.

Such a combination of therapeutic agents may be administered together or separately and, when administered separately, administration may occur simultaneously or sequentially, in any order. The amounts of the compounds or agents and the relative timings of administration will be selected in order to achieve the desired therapeutic effect. The administration in combination of a compound of the present invention with other therapeutic agents may be in combination by administration concomitantly in: (1) a unitary pharmaceutical composition including both compounds; or (2) separate pharmaceutical compositions each including one of the compounds. Such compounds include, but are not limited to, anti-inflammatory agents such as 5-amino salicylates, steroids, (e.g. methylprednisolone), azathioprine, 6-mercaptopurine, tacrolimus, and biologic agents that interfere with the actions of cytokines (e.g. tumor necrosis factor alpha) or the adhesion of inflammatory cells (e.g., natalizumab, sold under the tradename Tysabri®, or abatacept, sold under the tradename Orencia®). The combination of Compound (I) with steroids may result in the need for lower doses of the steroid with fewer steroid-associated adverse events. Alternatively, a combination may be administered separately in a sequential manner wherein one treatment agent is administered first and the other second. Such sequential administration may be close in time or remote in time.

Another aspect of the present invention relates to combination therapy comprising administering to the subject a therapeutically or prophylactically effective amount of the compound of the present invention and one or more other therapeutic agents including chemotherapeutics, radiation therapeutic agents, gene therapeutic agents, or agents used in immunotherapy.

### Pharmaceutical Compositions

In one aspect the present invention relates to pharmaceutical compositions comprising the compound of the present invention and one or more pharmaceutically acceptable carrier, diluent, or excipient. Another aspect of the invention provides a process for the preparation of a pharmaceutical composition including admixing the compound of the present invention with one or more pharmaceutically acceptable carrier, diluent, or excipient.

The manner in which the compound of the present invention is administered may vary. The compound of the present invention is preferably administered orally. Preferred pharmaceutical compositions for oral administration include tablets, capsules, caplets, syrups, solutions, and suspensions. The pharmaceutical compositions of the present invention may be provided in modified release dosage forms such as time-release tablet and capsule formulations.

The pharmaceutical compositions may also be administered via injection, namely, intravenously, intramuscularly, subcutaneously, intraperitoneally, intraarterially, intrathecally, and intracerebroventricularly. Carriers for injection may include 5% dextrose solutions, saline, and phosphate buffered saline.

The compositions may also be administered using other means, for example, rectal administration. The compounds may also be administered by inhalation, for example, in the form of an aerosol; topically, such as, in lotion form; transdermally, such as, using a transdermal patch (for example, by using technology that is commercially available from Novartis and Alza Corporation), by powder injection, or by buccal, sublingual, or intranasal absorption.

Pharmaceutical compositions may be formulated in unit dose form, or in multiple or subunit doses forms.

The administration of the pharmaceutical compositions described herein may be intermittent, or at a gradual, continuous, constant or controlled rate. The pharmaceutical compositions may be administered to a warm-blooded animal, for example, a mammal such as a mouse, rat, cat, guinea pig, rabbit, horses, dog, pig, cow, or monkey; but advantageously is administered to a human being.

### EXAMPLES

In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts, Protecting Groups in Organic Synthesis, 3^{rd} Edition, John Wiley & Sons, New York (1999)). These groups are removed at a convenient stage of the compound synthesis using methods that are readily apparent to those skilled in the art. The selection of processes as well as the reaction conditions and order of their execution shall be consistent with the preparation of compounds of the present invention.

The following examples are that demonstrate how the synthesis can be adapted to a chemical reactor, for large scale production of Compound 1, provided to illustrate the present invention, and should not be construed as limiting thereof. In these examples, all parts and percentages are by weight, unless otherwise noted.

The following abbreviations may be used, however, terms are believed to be used within the vernacular of those with ordinary skill.

THF tetrahydrofuran

NLT not less than

eq equivalent

NMT not more than

RX-1 Reactor number 1

RX-2 Reactor number 2

HPLC high pressure liquid chromatography

boc t-butoxy carbonyl

IPAc isopropyl acetate

### Nuclear Magnetic Resonance (NMR) Spectrometry

NMR spectra were collected on either a Varian Unity 300 MHz instrument or a Bruker 400MHz instrument equipped with an auto-sampler and controlled by a DRX400 console. Automated experiments were acquired using ICONNMR v 4.0.4 (build 1) running with Topspin v 1.3 (patch level 8) using the standard Bruker loaded experiments. For non-routine spectroscopy, data were acquired through the use of Topspin alone.

### Melting Point

Melting points were measured using digital scanning calorimetry.

### Process

The compounds may be prepared according to the following methods using commercially available starting materials and reagents. The preparation of Compound (I) is outlined in Scheme A.

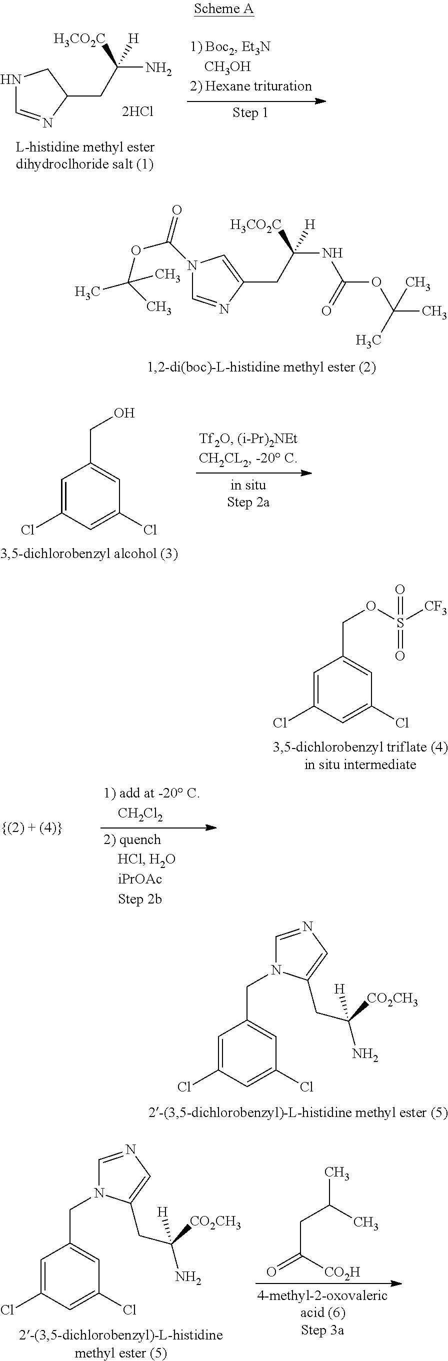

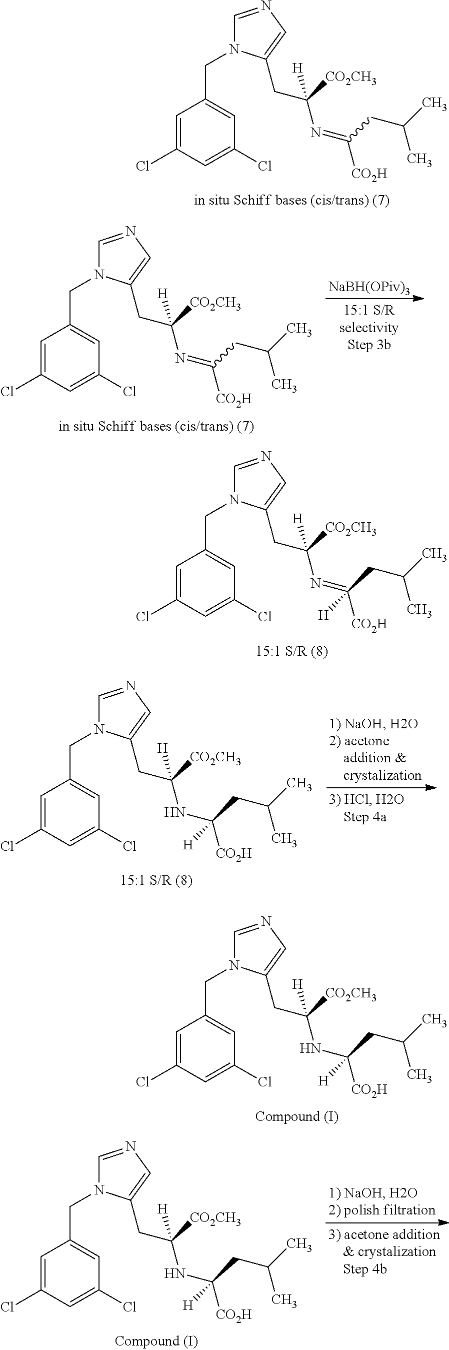

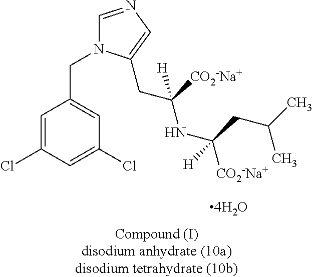

### Example I

As illustrated in Step 1, the L-histidine methyl ester dihydrochloride salt (1) was dissolved in methanol (5 mL/g) and cooled to 0° C. Triethylamine (2.2. equivalents) was added, and the reaction was held before addition of a solution of di-tert-butyl carbonate (2.2 equivalents) in methanol (2.5 mL/g of histidine methyl ester). The batch was stirred at 15° C. until the reaction reached completion. The L-histidine methyl ester was not detected during the completion of reaction analysis. After the reaction was deemed complete, methanol was removed under reduced pressure at 30° C. The concentrate was then partitioned between isopropyl acetate (6 mL/g histidine methyl ester) and water (4 mL/g histidine methyl ester). The isopropyl acetate layer was further washed with water (2 mL/g histidine methyl ester). The isopropyl acetate was then removed under reduced pressure at 30° C. The residual isopropyl acetate was removed by co-evaporation with heptane. The concentrate was then filtered and washed with heptane (8 mL/g histidine methyl ester) to remove the undesired 1′-regioisomer product. The resulting white to off white solid was dried under reduced pressure at 35° C. to provide the 3′,2-di-Boc protected intermediate (2) in 72% yield (Step 1).

### Example II

The second step consists of two parts: (a) an alkylation reaction using the triflate derivative (4) of the 3,5-dichlorobenzyl alcohol (3) as the alkylation agent (Step 2a); and (b) the removal of a Boc group using HCl (Step 3) to produce the N1′-alkylated L-histidine ester (5) (Step 2b).

Charges for Step 2a are based on the charge of the 3′,2-di-Boc protected intermediate (2) to be used.

Dichloromethane (2.8 mL/g of 2) was cooled to less than −20° C. and triflic anhydride (1.1 equivalents) added. A solution of the dichlorobenzyl alcohol (1.1 equivalents) and diisopropylethylamine (1.1 equivalents) in dichloromethane (2.8 mL/g 2) was then transferred to the reaction mixture, maintaining the internal temperature below −20° C. After a hold time of one hour, a solution of (2) in dichloromethane (1.75 mL/g of 2) was charged to the reaction, maintaining the internal temperature below −20° C. The reaction was allowed to slowly come to ambient temperature and held for a minimum of 10 hours. Analysis of the mixture by HPLC indicated a complete reaction. A solution of potassium phosphate monobasic (0.384 g/g of 2) in water (4.7 mL/g of 2) was charged to the reaction. The reaction was partitioned, and the organic layer was washed with an aqueous solution of sodium bicarbonate (0.469 g/g of 2 in 4.7 mL water/g of 2). After partitioning, the organic layer was washed with water (4.7 mL/g of 2). The dichloromethane was then removed under reduced pressure at 30° C.

The concentrate was then dissolved in isopropyl acetate (4 mL/g of 2) and the mixture was cooled to 0° C. Concentrated hydrochloric acid (1.07 mL/g of 2) was charged at the mixture was allowed to come slowly to ambient temperature over 15 hours. After an in-process analysis by HPLC indicated complete reaction, the slurry was filtered, and the filter cake was washed with isopropyl acetate (3.5 mL/g of 2) and then heptane (2.3 mL/g of 2). The product was then dried under reduced pressure at 30° C. to provide the desired product as a white to off-white solid in 65-80% yield.

### Example III

These steps involve the synthesis of the NaBH(OPiv)₃ reagent, a subsequent reductive amination with 4-methyl-2-oxovaleric acid (6) via Schiff Base mixture (7) (Step 3a) to yield ester (8) (Step 3b). The ratio of the stereoisomers in ester (8) was 15:1 at the conclusion of the reaction (<1% starting material remained).

For a publication of the synthesis of the reagent sodium tri(pivaloyloxy)borohydride, see “A highly stereoselective reductive amination of 3-ketosteroid with amines: an improved synthesis of 3a-aminosteroid”, Khan, S. N., Bae, S.-Y. and Kim, H.-S. Kim, Tet. Lett., 2005, 46, 7675-7678, herein incorporated by reference with regard to such synthetic teaching.

Preparation of NaBH(OPiv)₃ Reagent:

Pivalic acid (PA) was dissolved in THF (4.65 mL/g PA). The solution was cooled to 0° C. and sodium borohydride (12.32 g/mole of PA) was added. The mixture was then warmed to 60° C. and held for 16 hours. The THF was removed under reduced pressure at 30° C., and residual THF was further removed by co-distillation with heptane (1.5 mL/g PA). The reagent was then diluted with heptane (2.64 mL/g PA).

Reductive Amination

2-Propanol (0.61 mL/g PA) was then added to the NaBH(OPiv)₃ Reagent. The dihydrochloride salt product resulting from Example 2 was then added (0.327 g/g of PA). The reaction was cooled to −8° C. and the 4-methyl-2-oxopentanoic acid sodium salt (1.137 g/g of dihydrochloride) was added. The mixture was held for a minimum of 8 hours at −8° C. until the in-process analysis indicated a complete reaction.

An aqueous solution of hydrochloric acid (1.08 mL HCl/g of sodium borohydride) and water (2 mL/g of sodium borohydride) was added to the reaction, and the mixture was concentrated under reduced pressure at 35° C. Removal of traces of 2-propanol was accomplished co-distillation with additional heptane. The concentrate was then partitioned between isopropyl acetate (6 mL/g of dihydrochloride) and water (5.1 mL/g of dihydrochloride). An aqueous solution of hydrochloric acid (0.7 mL/g of dihydrochloride) in water (4.5 mL/g dihydrochloride) was then added to the organic layer. The organic layer was re-extracted a second time with a similar aqueous solution of hydrochloric acid. The aqueous layers were combined, and extracted with isopropyl acetate (1.5 mL/g of dihydrochloride). After partitioning, the pH of the aqueous layer was then adjusted to pH=6 with an aqueous solution of potassium carbonate (1.88 g/g of dihydrochloride in 3.5 mL water/g of dihydrochloride). The batch was then extracted with isopropyl acetate (6 mL/g dihydrochloride). The aqueous layer was then re-extracted twice with isopropyl acetate (1.8 mL/g dihydrochloride). The combined organic layers were then washed with a brine solution (10%; 3 mL/g dihydrochloride). The organic layer was then concentrated under reduced pressure at 35° C. Residual isopropyl acetate was removed by codistillation with a mixture of heptane and ethanol (1:1). Codistillation was continued until in-process analysis indicated complete removal of isopropyl acetate.

### Example IV

The saponification of ester (8) with sodium hydroxide was completed by dissolving ester (8) in ethanol (0.25 mL/g of dihydrochloride) and cooling the solution to 0° C. An aqueous solution of water (1.5 mL/g of dihydrochloride) and sodium hydroxide (0.126 g/mL of water) was then added, and the reaction was stirred for a minimum of 1 hour at 0° C. The reaction was then warmed to 20° C. and stirred until an in-process analysis indicated complete reaction. The solution was then concentrated under reduced pressure at 35° C. The disodium salt was directly isolated from the saponification by addition of acetone (11 mL/g of water) and filtration of the solids. The disodium salt was then acidified with hydrochloric acid to give diacid, Compound (I). The disodium salt was dissolved in water (5 mL/g salt) and acidified to pH 3-4. The diacid was then isolated by filtration, and the filter cake was washed with water to remove incipient hydrochloric acid. (Step 4a)

### Example V

The diacid (Compound (I)) was then further slurried in water (5 mL/g of salt input and refiltered. The filter cake was again rinsed with water (2 mL/g) and heptane (2 mL/g). The disodium salt was again formed by addition of sodium hydroxide to produce a solution that can be polish filtered. Slow addition of acetone anti-solvent (13 mL/g) to this solution effected crystallization of purified disodium dicarboxylate tetrahydrate (10b) (Step 4b). The mixture was aged for not less than 8 hours before filtration and a subsequent rinse of the product with acetone (2 mL/g) Depending on how the step and drying are conducted, purified disodium dicarboxylate anhydrate (10a), disodium dicarboxylate tetrahydrate (10b), or disodium dicarboxylate tetrahydrate, acetone solvate (10c) can be obtained. If further purification is necessary or desired, the final product can be dissolved in water and re-precipitated using acetone (11 mL/g) as the antisolvent. For the disodium dicarboxylate tetrahydrate (10b) drying is accomplished under partial vacuum at 30° C.

As will be appreciated, modifications in this step may produce purified disodium dicarboxylate anhydrate (10a), disodium dicarboxylate tetrahydrate (10b), disodium dicarboxylate tetrahydrate (10b), or disodium dicarboxylate tetrahydrate, acetone solvate (10c).

### Example 1

### Preparation of the diBoc-Histidine Methyl Ester (2)

### (S)-4-(2-tert-butoxycarbonylamino-2-methoxycarbonyl-ethyl)-imidazole-1-carboxylic acid tertbutyl ester

Charged L-histidine methyl ester dihydrochloride (1.0 eq) and methanol to RX-1. Cooled to 0° C. and add 2.2 eqs of triethylamine maintaining a temperature of NMT 10° C. Mixed at 0° C. for NLT 2 hours. Charged 2.2 eqs of di-tert-butyl dicarbonate and methanol to RX-2. Mixed to dissolve solids at 10° C. Transferred the Boc anhydride solution to RX-1 over NLT 1 hour maintaining RX-1 at NMT 20° C. Adjusted RX-1 internal temperature to 20° C., mix for NLT 4 hours and sample for reaction completion until NLT 1% L-Histidine methyl ester starting material is remaining. Distilled the contents of RX-1 under vacuum until no more distillate is observed. Maintained jacket temperature below 35° C. Charged distilled water and isopropyl acetate to RX-1. Mixed for NLT 15 minutes, allowed to settle and separated the aqueous layer. Charged distilled water to RX-1. Mixed for NLT 15 minutes, allowed to settle and separated the aqueous layer. Distilled the contents of RX-1 under vacuum to concentrate. Maintained a jacket temperature of NMT 45° C. Charged heptane over NLT 1 hour with a jacket temperature of 30° C. Charged isopropyl acetate and heat to 35° C. to dissolve solids. Distilled the contents of RX-1 under vacuum. Maintain a jacket temperature of NMT 37° C. Charged heptane to RX-1. Distilled the contents of RX-1 under vacuum to concentrate. Maintained a jacket temperature of NMT 37° C. Charged heptane to RX-1. Distilled the contents of RX-1 under vacuum to concentrate. Maintained a jacket temperature of NMT 37° C. Mixed the contents of RX-1 for 6 hours at an internal temperature of 20° C. Filtered the product, washing each load with heptane. Submitted sample for HPLC purity analysis. Record percent purity of Diboc Histidine methyl ester. Dried in tray dryer under vacuum for NLT 24 hours. Target 35° C. with a maximum temperature of NMT 40° C.

Isolated 8.45 Kgs (75.8% step yield)

HPLC: 99.7%.

### Example 2

### Preparation of (S)-2-amino-3-[3-(3,5-dichlorobenzyl)-3H-imidazol-4-yl]Propionic Acid Methyl Ester (5)

The second step consists of two parts: (a) an alkylation reaction using the triflate derivative (4) of the 3,5-dichlorobenzyl alcohol (3) as the alkylation agent; and (b) the removal of the Boc groups using HCl to produce the N1′-alkylated L-histidine ester (5). Step 2a

Charged methylene chloride to a portable reactor and cool to less than −20° C. Charged 1.1 eqs triflic anhydride to the portable reactor maintaining the temperature below −20° C.

In a separate reactor, charged 3,5-dichlorobenzyl alcohol (3) (1.1 eqs), methylene chloride and N,N-diisopropylethylamine (1.13 eqs) and mix for NLT 1 hour to dissolve solids. Cooled the solution to less than −10° C. and slowly transfer to the triflic anhydride solution in the portable reactor maintaining the internal temperature at less than −20° C., producing (4). Mixed the reaction solution at less than −20° C. for NLT 1 hour. Step 2b

In a separate reactor, dissolved (2) from Example 1 (1 eq) in methylene chloride. Transferred (2) to the reactor containing the Triflic anhydride mixture. Warmed reaction to NMT 35° C. and stir NLT 10 h. Sample reaction until NMT 1% (2) remains. Quenched the reaction mixture with a potassium phosphate solution KH₂PO₄ and water. Mixed for NLT 30 minutes, settled for NLT 1 hour and separated the aqueous layer. Washed the organic layer with a sodium bicarbonate solution. Mixed for NLT 30 minutes, settle for NLT 1 hour and separated the aqueous layer. Distilled the organic layer under vacuum. Maintained a jacket temperature of NMT 35° C. Diluted the residue with isopropyl acetate, mix to dissolve solids and cool to 0° C. Charged conc. HCl to the solution maintaining an internal temperature of NMT 5° C. and mix for NLT 1 hour. Heat the reaction mixture to 20° C. over NLT 3 hours and mix for NLT 15 hours. Pull sample for reaction completion until NMT 1% mono boc compound remaining. Filter the product (5) and wash with isopropyl acetate and heptane. Dried in tray dryer under vacuum for NLT 24 hours. Target 30° C. with a maximum temperature of NMT 35° C. Dried until constant weight achieved (NMT 1% change).

Isolated 5.76 Kgs (82.4% step yield)

HPLC: 96.24% with isomer of >99%

### Example 3

These steps involve the synthesis of the NaBH(OPiv)₃ reagent, a reductive amination of (5) with 4-methyl-2-oxovaleric acid (6) to yield Schiff Base mixture (7) and reaction of (7) to yield ester (8). The ratio of the stereoisomers in ester (8) was 15:1 at the conclusion of the reaction (<1% starting material remained). These steps were performed as follows.

Charged with THF as the solvent (4.65 mL/g pivalic acid) and trimethylacetic acid (pivalic acid) (14 eqs) to RX-1. Cooled to less than −10° C. and charge sodium borohydride (4.7 eqs) portion wise. Raised the temperature to 20° C. and mixed for NLT 1 hour. Heated to reflux and hold for NLT 16 hrs. Tested reagent for activity by performing use test of BH(OPiv)3. Distilled the contents of RX-1 under vacuum to a volume. Maintained a jacket temperature of NMT 45° C. Charged heptane and distill under vacuum to a volume. Maintained a jacket temperature of NMT 45° C. Charged heptane and distill under vacuum. Maintained a jacket temperature of NMT 45° C. Charged heptane and distill under vacuum. Maintained a jacket temperature of NMT 45° C.

Added isopropyl alcohol to RX-1, approximately 22% of total heptanes. Charged ester (5) (1.0 eqs) and 4-methyl-2-oxopentanoic acid sodium salt (6) (3.0 eqs) to RX-1. Mixed for NLT 30 minutes at −5° C. Pulled Sample to test for starting material. Reaction is complete when NMT 5% (5) is present.

Charged distilled water. Prepared a 20% HCl solution with distilled water and hydrochloric acid and transfer to the solution in RX-1. Distilled the contents of RX-1 under vacuum. Maintained a jacket temperature of NMT 45° C. Charged heptane and distill under vacuum. Maintained a jacket temperature of NMT 45° C. Charged IPAc and distilled water to RX-1. Mixed for NLT 15 minutes, settled for NLT 15 minutes and separated the aqueous layer that contains product. Charged flask with heptane and mix for NLT 30 minutes. Prepared a 5% HCl solution with water and HCl and transfer to RX-1. Mixed for NLT 15 minutes, settled for NLT 30 minutes and separated the lower (product) layer. Transferred the remaining 5% HCl solution to RX-1. Mixed for NLT 15 minutes, settle for NLT 15 minutes and separate the lower (product) layer. Extracted the product solution in by charging IPAc and heptane. Mixed for NLT 15 minutes, settled for NLT 15 minutes and separated the lower (product) layer. Extracted the product solution in RX-1 by charging IPAc and heptane to RX-1. Mixed for NLT 15 minutes, settled for NLT 15 minutes and separated the lower (product) layer. Charged IPAc to RX-1. Prepared a 30% K₂CO₃ solution in distilled water and use to adjust the pH of RX-1 to 6. Mixed RX-1 for NLT 15 minutes, settled for NLT 15 minutes and separated the lower layer (waste). Charged distilled water to RX-1, mix for NLT 15 minutes, settled for NLT 15 minutes and discarded the lower layer. Distilled the contents of RX-1 under vacuum. Maintained a jacket temperature of NMT 45° C. Charged heptane to RX-1 and distilled under vacuum. Maintained a jacket temperature of NMT 45° C. Charged heptane to RX-1 and distilled under vacuum. Maintained a jacket temperature of NMT 45° C. Cooled to 0° C. Prepared a 25% sodium hydroxide solution by charging distilled water and 50% sodium hydroxide. Cooled to 0° C. Slowly transferred the 25% sodium hydroxide solution while maintaining the internal temperature at NMT 20° C. Cooled to 0° C., charged ethanol and mixed for NLT 1 hour. Pulled sample and test for starting material. Reaction is complete when NMT 1.0% methyl ester acid (8) is present. Added acetone slowly to the reactor using either an addition funnel or a transfer pump. Slowly added the remaining ACE and age the reaction for a minimum 8 hours at 20±5° C. Filtered and wash solids with acetone.

HPLC by achiral and chiral HPLC analysis. If the chemical purity of the product is less than 98.5% and/or the chiral purity is less than 98.5%, repeated recrystallization steps.

### Example 4

The saponification of ester (8) with sodium hydroxide was completed. The disodium salt was directly isolated from the saponification and acidified with hydrochloric acid to give diacid (9), which was isolated by filtration.

The diacid (9) was further slurried in water, and the disodium salt was again formed by addition of sodium hydroxide to produce a solution which was polish filtered. This solution was cooled with slow addition of filtered acetone anti-solvent to effect crystallization of purified disodium dicarboxylate tetrahydrate (10b). Depending on how this step is conducted, purified disodium dicarboxylate anhydrate (10a), disodium dicarboxylate tetrahydrate (10b), disodium dicarboxylate tetrahydrate (10b), or disodium dicarboxylate tetrahydrate, acetone solvate (10c) can be obtained.

### Characteristics

### X-Ray Powder Diffraction (XRPD)

X-Ray Powder Diffraction patterns were collected using a PANalytical X′Pert Pro diffractometer using CuKα radiation. An incident beam of CuKα radiation was produced using an Optix long, fine-focus source. An elliptically graded multilayer mirror was used to focus the CUKα X-rays of the source through the specimen and on to the detector. Data were collected and analyzed using X′Pert Pro Data Collector software (v. 2.2b). Prior to analysis, a silicon specimen (NIST SRM 640c) was analyzed to verify the Si 111 peak position. Each specimen was sandwiched between 3 μm thick films, analyzed in transmission geometry, and rotated to optimize orientation statistics. A beam-stop was used to minimize the background generated by air scattering. A helium atmosphere and anti-scattering extension were not used. Soller slits were used for the incident and diffracted beams to minimize axial divergence. Diffraction patterns were collected using a scanning position-sensitive detector (X′Celerator) located 240 mm from the specimen.

### Differential Scanning Calorimetry (DSC)

DSC data were collected on a TA Instruments Q2000. The instrument was calibrated for energy and temperature calibration using certified indium. Each sample was placed into an aluminum DSC pan and the weight accurately recorded. The pan was covered with a lid, and the lid was crimped. A weighed, crimped aluminum pan was placed on the reference side of the cell. The sample cell was equilibrated at −30° C. and heated under a nitrogen purge at a rate of 10° C./minute, up to a final temperature of 250° C.

### Refractive Index Determination

Refractive index determination was performed using a Leica DM LP microscope. A single, sub-stager polarizer was used to view the samples. Samples were prepared in a glass slide with a coverslip and dispersed in various Cargille refractive index oils. The movement of the Becke line was observed while defocusing the sample.

### Polarized Light Microscopy (PLM)

Samples were studied on a Leica DM LP microscope with a digital video camera for image capture (Spot Insight color camera model 3.2.0). A small amount of each sample was placed on a glass slide, mounted in immersion oil and covered with a glass slip, the individual particles being separated as well as possible. The sample was viewed with appropriate magnification and partially polarized light, coupled to a A false-color filter.

### Particle Size Analysis

Particle size analysis was acquired using a Malvern Instruments MS2000 equipped with a Hydro2000 μP dispersion unit. Data was collected and analyzed using Mastersizer 2000 v 5.1 software, using volume based measurements. NIST traceable glass beads were used as the reference standard.

### Example 5

### XRPD

XRPD analysis was performed for several salt samples herein described. The

XRPD sample information and results are summarized in Table 1:

TABLE 1

| Sample | Results |
|---|---|
| Batch A | Crystalline |
| Batch B | Crystalline |
| Batch C | Crystalline |
| Batch D | Disordered Crystalline |
| | Mixture: Crystalline & Amorphous |

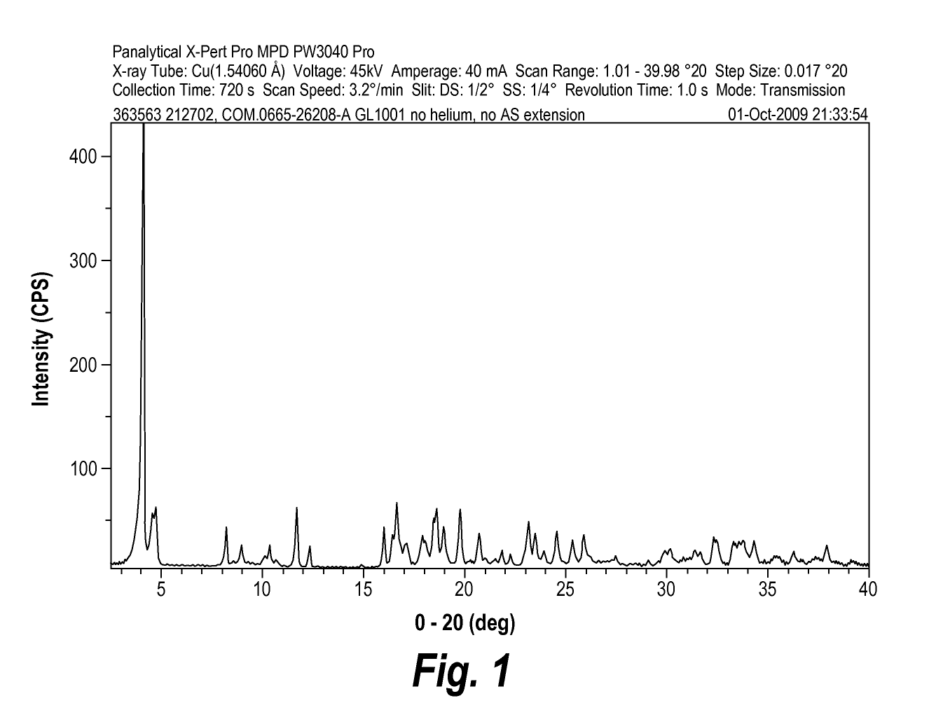

As illustrated in FIGS. 1, 2, and 3, the XRPD patterns of Batches A, B, and C exhibit relatively sharp peaks, indicating the samples are composed of crystalline material.

The pattern of Batch D exhibits relatively sharp peaks on a diffuse scattering background indicating this sample is composed of disordered crystalline material or a mixture of crystalline and amorphous material, see FIG. 4. The partial amorphous character is associated with capsule failure, possibly resulting from dehydration of the gelatin capsules.

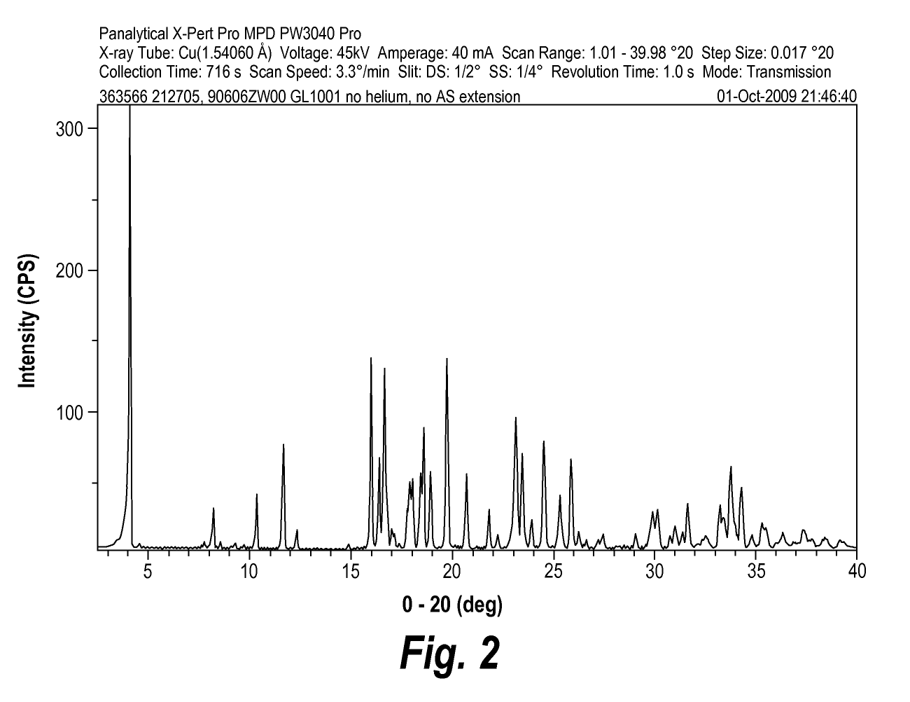

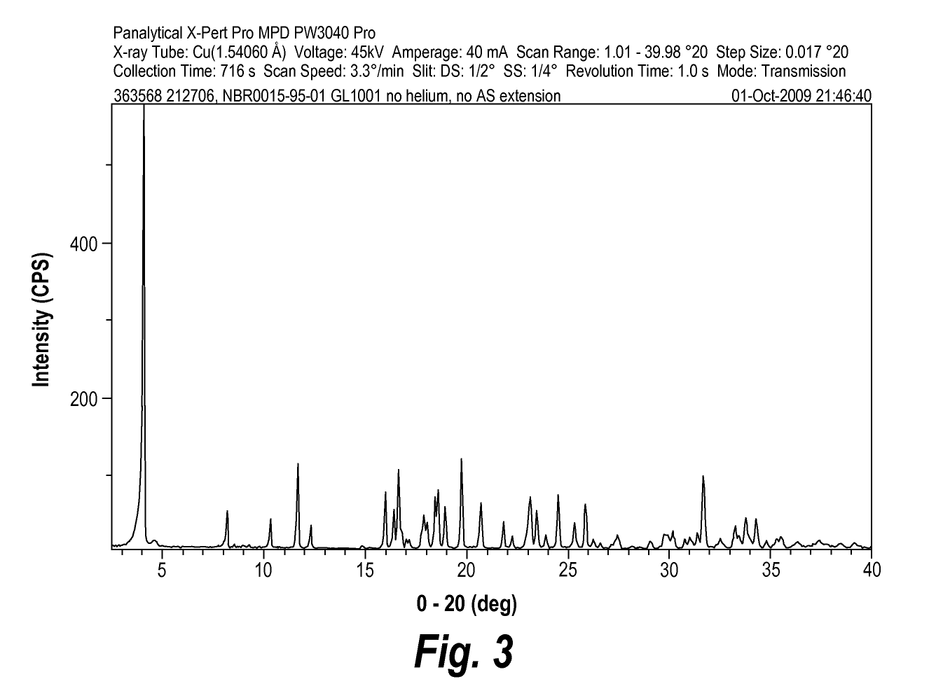

The XRPD patterns of Batches A (FIG. 1), B (FIG. 2), and C (FIG. 3) are visually similar in terms of peak position in degrees 26, suggesting they are composed of the same form or mixture of forms. Additional peaks at approximately 4.7 and 9.0° 2θ were observed in the pattern of Batch A compared to Batch B or Batch C, indicating Batch A may contain an additional phase. The additional peaks at 4.7 and 9.0° 2θ were also observed in the pattern of Batch D.

### Example 6

### Physical Properties

The preferred crystal patterns possess many excellent properties for a useful pharmaceutical preparation. Among these are high water solubility (as used herein about 100 mg/mL), excellent long-term stability (as used herein % degradation over about 7 years), high density for convenient capsule or tablet size (as commonly used in the art) and good stability of the drug product when formulated in capsules (as commonly used in the art).

One form (FIG. 4) proved to be unsuitable for gelatin capsule formulation, yielding degraded capsules within one month of room temperature storage.

### Differential Scanning Calorimetry (DSC)

The DSC sample information and results are summarized in Table 2

TABLE 2

| Sample | Results (temps rounded to nearest whole ° C.) |
|---|---|
| Batch A | Sharp endotherm 87° C. |
| | Endotherms: 99, 103, 116° C. |
| Batch B | Sharp endotherm 91° C. |
| | Endotherms: 98, 106, 115° C. |
| Batch C | Sharp endotherm 90° C. |
| | Endotherms: 94, 102, 116° C. |
| Batch D | Broad endotherm 30° C. |
| | Exotherm: 78° C. |
| | Endotherms: 89, 123° C. |

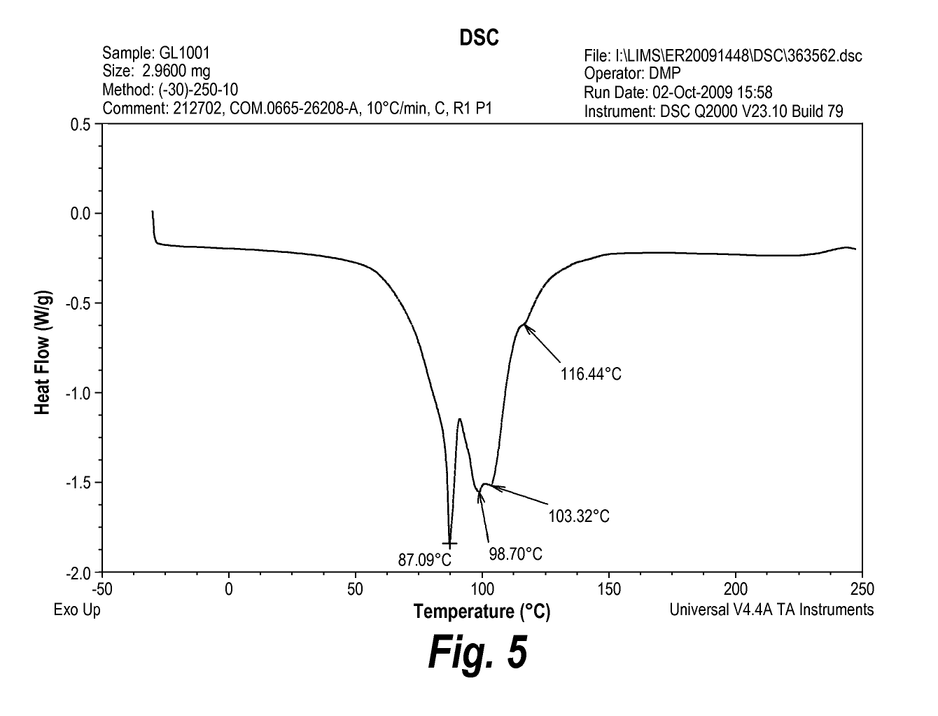

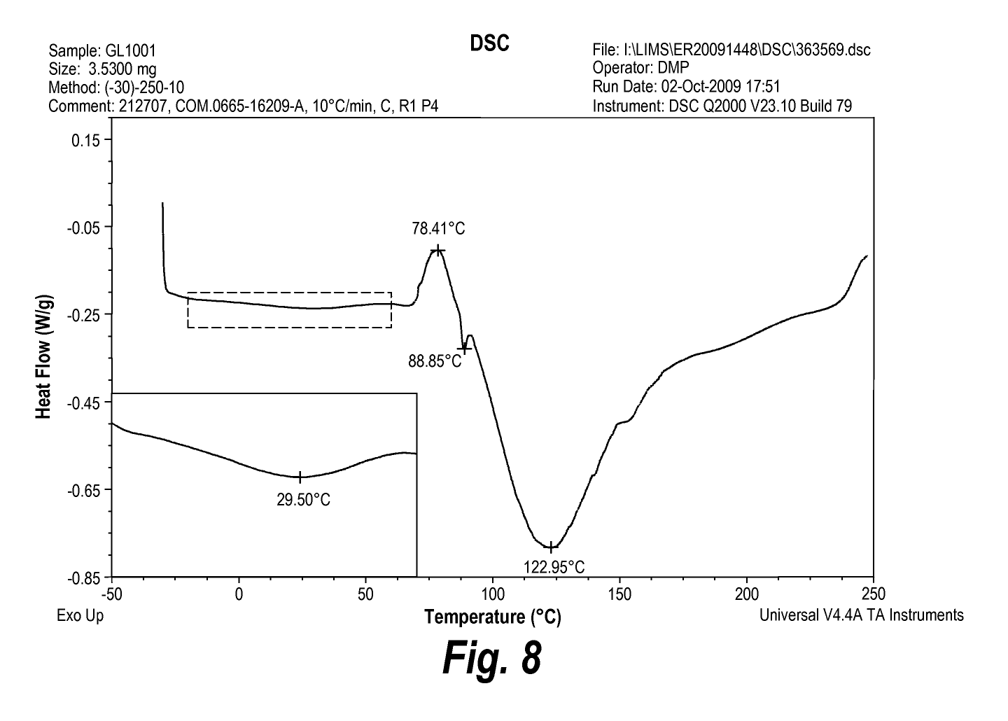

FIGS. 5, 6, 7, and 8 present DSC thermograms for the four samples, respectively. The DSC thermograms of Batches A, B, and C exhibit similar thermal events. The sharp endotherm with a maximum at approximately 87-91° C. observed in each of the thermograms is typical of a melt. This event is followed by several endothermic events with maxima ranging from approximately 94° C. to approximately 116° C.

The thermogram for Batch D exhibits a broad endotherm with a maximum at approximately 30° C. that is likely due to loss of volatiles. The exotherm displayed in the DSC thermogram at approximately 78° C. suggests crystallization, or that the sample may become less disordered after desolvation. This event is closely followed by endotherms with maxima at approximately 89 and 123° C., respectively.

The nature of the events noted in the DSC thermograms may be verified with the aid of other techniques, such as thermogravimetry (TG), hot-stage light microscopy, and/or variable temperature X-ray powder diffraction (VT-XRPD).

The product of Batch A was used to develop the particle size. Three dispersants, 0.01% (w/v) Tween 20 in water, 0.1% (w/v) Lecithin in Isopar G, and 0.1% (w/v) SPAN 85 in hexane, were evaluated for use. The sample dissolved in the 0.01% (w/v) Tween 20 in water and the dispersant was deemed unsuitable.

The sample dissolved when suspended in 0.01% (wlv) Tween 20 in water; therefore, this dispersant was deemed unsuitable. The sample suspended with shaking in 0.1% (wlv) Lecithin in Isopar G and 0.1% (w/v) SPAN 85 in hexane; however, the material dropped out of suspension relatively quickly in both dispersants. These two dispersants were further evaluated using polarized light microscopy (PLM). The sample appeared to be slightly better dispersed in the higher viscosity 0.1% (w|v) Lecithin in Isopar G; therefore, 0.1% (w/v) Lecithin in Isopar G was selected as the appropriate dispersant.

The sample absorption value was determined by reprocessing a measurement using various absorption values. When observing trend graphs for the d10, d50 and d90 particle size distribution vs. recirculation time, there is noticeable leveling off, which occurs after approximately 4-5 minutes, suggesting that the agglomerates are dispersed to the highest degree possible after approximately 5 minutes of recirculation.

Striations in the larger particles suggest that they may be clusters of needle-like particles. Pressure applied using a needle point during PLM observation of the sample dispersed in 0.9% (w|v) Lecithin in Isopar G showed that agglomerates could be dispersed by moderate pressure and agglomerates and larger primary particles could be broken; therefore, no benefit was shown by using a dispersant with a higher surfactant concentration.

A comparison of the particle size distributions from a recirculation evaluation with the particle size distribution using 50% sonication suggested that even mild sonication resulted in particle attrition; therefore, a recirculation time of three minutes without sonication was chosen for the final method.

The repeatability of the method was evaluated by making three replicate measurements using the final method conditions (Table 3).

TABLE 3

| Record | d10 (μm) | d50 (μm) | d90 (μm) |
|---|---|---|---|
| 1 | 89.4 | 262.4 | 505.0 |
| 2 | 92.6 | 259.3 | 501.9 |
| 3 | 93.3 | 257.2 | 494.6 |
| Mean | 91.8 | 259.6 | 500.5 |
| St Dev | 2.1 | 2.6 | 5.3 |
| % RSD | 2.3 | 1.0 | 1.1 |

Expressed as percentage (10, 50, 90) of the particles by volume being less than the indicated particle size in μm.

The relative standard deviations for the d10, d50 and d90 were 2.3096, 1.01% and 1.0696, all of which fall within the USP recommendation of ≦15%, ≦10%, and ≦15% for the d10, d50, and d90, respectively. Photomicrographsof a sample taken following the repeatability analysis indicated that the sample was well dispersed and showed no particle attrition when compared to photomicrographs taken in mineral oil.

The final unvalidated method conditions selected for determining the particle size were:

Sample refractive index: 1.56

Sample absorption: 0.1

Dispersant: 0.1% (w|v) Lecithin in Isopar G

Dispersant refractive index: 1.42

Pump speed: 2000 rpm

Recirculation time: 3 minutes

Sample measurement time: 10 sec.

Background measurement time: 20 sec.

Model: general purpose

Sensitivity: normal

### Particle Size Sample Analysis

One particle size measurement of each of the four samples was taken (Table 4) using the final method conditions. The results for Batch A were consistent with those collected for this sample during method development. Particle size distributions for the other three lots, Batches B, C, and D, varied with d10 values ranging from approximately 64.2 pm to 90.5 μm, d50 values ranging from approximately 181.7 μm to 282.5 μm, and d90 values ranging from approximately 364.2 μm to 595.0 μm.

TABLE 4
Particle Size

| Sample | d10 (μm) | d50 (μm) | d90 (μm) |
|---|---|---|---|
| Batch A | 90.5 | 247.9 | 478.9 |
| Batch B | 73.0 | 230.9 | 454.4 |
| Batch C | 77.3 | 181.8 | 364.2 |
| Batch D | 64.2 | 282.5 | 595.0 |

Expressed as percentage (10, 50, 90) of the particles by volume being less than the indicated particle size in μm.

### Salt Forms

The stoichiometry of the salts comprised in the present invention may vary. For example, it is typical that the molar ratio of acid to Compound (I) is 1:2 or 1:1, but other ratios, such as 3:1, 1:3, 2:3, 3:2 and 2:1, may be possible and are likewise included in the scope of the present invention. Depending upon the manner by which the salts described herein are formed, the salts may have crystalline structures that occlude solvents that are present during salt formation. Thus, the salts may occur as hydrates and other solvates of varying stoichiometry of solvent.

### Biology

2-[1-(S)-carboxy-2(S)-[3-(3,5-dichloro-benzyl)-3H-imidazol-4-yl]-ethylamino]-4-methyl-pentanoic acid, herein also referred to as Compound (I), is described in U.S. Pat. No. 6,632,830, U.S. Pat. No. 7,045,532, and WO 00/066104, each herein incorporated by reference in their entirety. Additionally, Compound (I) is discussed in “Substrate-Based Design of the First Class of Angiotensin-Converting Enzyme-Related Carboxypeptidase (ACE2) Inhibitors”, Dales, N. A., et al., J. Amer. Chem. Soc., 2002, 124, 11852-11853, incorporated by reference. As reported by Byrnes et al., Effects of the ACE2 inhibitor GL1001 on acute dextran sodium sulfate-induced colitis in mice, Inflamm. Res. (2009) 58:819-827, incorporated by reference, Compound (I) is a potent and selective ACE2 inhibitor with anti-inflammatory activity in the digestive tract. Doses of Compound (I) ameliorated DSS-induced disease activity, including rectal prolapsed and intestinal bleeding. Colon pathology and myeloperoxidase activity were also markedly attenuated by treatment with Compound (I), with a profound effect observed in the distal segment. Compound (I) provides therapeutic potential for inflammatory bowel disease.

Two-way ANOVA revealed effects of Compound (I) treatment on body weight in animals pre-exposed to dextran sodium sulfate (DSS, reagent grade from MP Biochemicals, Solon, Ohio) in drinking water. Animals having received normal drinking water (no DSS) followed by repeated b.i.d. vehicle treatment (vehicle group) showed a slight increase in body weight across experimental days 5-15. Exposure to DSS prior to vehicle treatment (vehicle+DSS group) produced a time-dependent decrease in body weight that recovered by day 15. This body weight loss and recovery was similar in animals having received repeated doses of Compound (I) (300 mg/kg). In contrast, while body weight loss in sulfasalazine-treated animals was similar to both of these groups, it did not recover to the same extent by day 15. Animals treated with Compound (I) (30 and 100 mg/kg) exhibited the largest weight reduction with maximum effects observed on day 9. Both groups experienced body weight gain after this time, with 30 mg/kg producing compete recovery by day 15.

Disease activity index (DAI), namely rectal prolapsed, stool consistency, and fecal occult blood) was assessed every other day throughout the experiment. By experimental day 7, significant DSS-induced disease began to emerge, and trends toward recovery were observed by day 11. Statistical analyses were conducted on data obtained on the days of peak severity: day 7 (presence of fecal blood and diarrhea) and day 9 (rectal prolapse). All DAI data were analyzed by one-way ANOVA. Compared to non-DSS controls, DSS treatment markedly elevated scores for fecal occult blood, stoll consistency, and rectal prolapsed. These effects were attenuated by Compound (I) (300 mg/kg). For rectal prolapsed and bleeding, sulfasalazine (150 mg/kg) was similarly efficacious. For lower doses of Compound (I), namely 30 or 100 mg/kg, no effects were observed on any DAI parameter (all P>0.05).

On experimental day 15, colon was obtained from each animal and length was measured. DSS treatment resulted in 11% colon shortening: vehicle treated (no DSS) 80.87±3.24 mm, vehicle+DSS-treated 71.62±0.97 mm. One-way ANOVA indicated that this effect was statistically significant and was reversed by high dose Compound (I) and sulfasalazine. No effects of 30 or 100 mg/kg on DSS-induced colon shrinkage were observed (P>0.05 for all). Total histopathology score (inflammation+glandular epithelial loss+mucosal erosion) for each treatment group is represented with a maximum obtainable score of 15. One-way ANOVA indicated significant DSS_induced damage (vs. non-DSS control) in each region studied, with the most damage occurring in the distal colon. Treatment with Compound (I) (300 mg/kg) significantly attenuated DSS-induced damage in the distal colon, providing a protective effect on inflammation, edema, gland loss, and erosion.

Specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A process for preparing Compound (I) 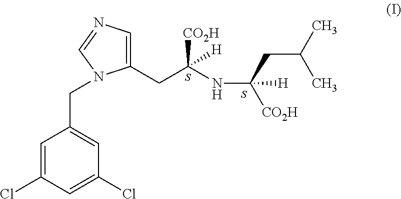
or a pharmaceutically acceptable salt, or hydrate, or solvate thereof, comprising an enantioselective reductive amination.

2. The process of claim 1, further comprising the use of pivaloyloxyborohydride or a salt thereof.

3. The process of claim 2, wherein the pivolyl borohydride is used in a 15:1 ratio.

4. The process of claim 2, further comprising a subsequent acetone wash.

5. The product prepared by claim 1.

6. A process for preparing Compound (I), 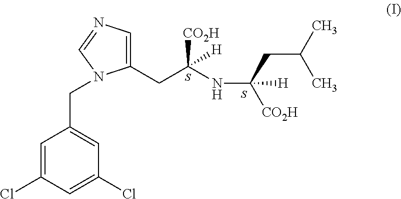
or a salt, hydrate, solvate, or polymorph thereof, comprising the steps of:
(a) acylating a compound of formula (II) 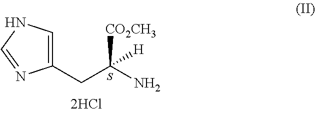
or a salt thereof, with di-tert-butyl dicarbonate—(Boc)₂O to form the compound of formula (III) 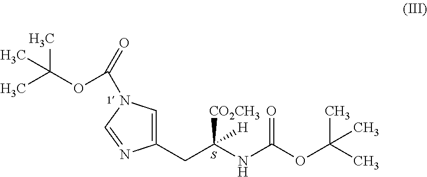
(b) reacting 3,5-dichlorobenzyl alcohol of formula (IV) with triflic anhydride and diisopropylethylamine base 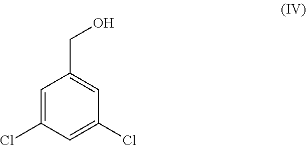
to form labile 3,5-dichlorobenzyl triflate of formula (V) 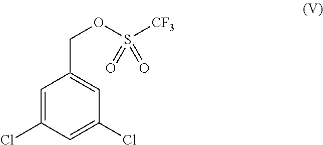
(c) coupling of the compound of formula (III) with 3,5-dichlorobenzyl triflate of formula (V) to produce intermediate compound of formula (VI) 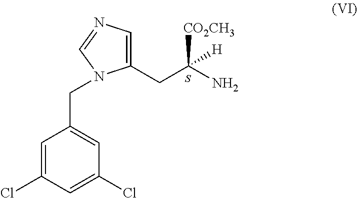
(d) coupling of the compound of formula (VI) with 4-methyl-2-oxovaleric acid to form Schiff base mixture of formula (VII) 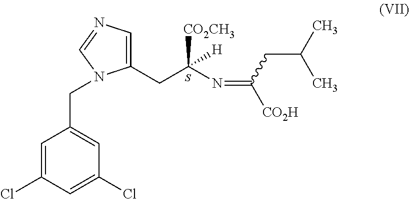
(e) reductive amination of the Schiff base mixture of formula (VII) in the presence of sodium tri(pivaloyloxy)borohydride to form a compound of formula (VIII) as a mixture of diastereoisomers 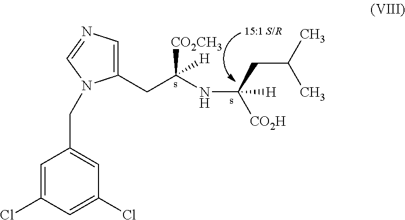
(f) saponification of the compound of formula (VIII) with aqueous sodium hydroxide, followed by acidification with aqueous hydrochloric acid and isolating the resulting solid product.

7. The process of claim 6, wherein the salt of compound (I) is a monosodium salt, a disodium salt, a monopotassium salt, a dipotassium salt, a monoammonium salt or a diammonium salt, or a combination thereof containing sodium, potassium or ammonium counterions, or a hydrate of solvate thereof.

8. The process of claim 7, wherein the salt of compound (I) is a disodium salt of formula (IX) 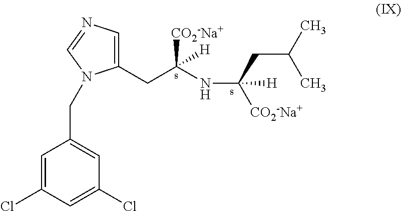
or a hydrate or solvate thereof.

9. The process of claim 7, wherein the salt of compound (I) is a disodium tetrahydrate salt of formula (X)

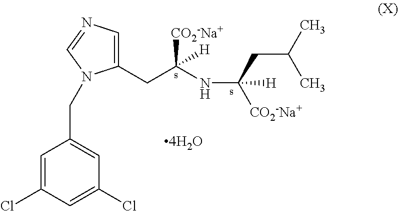

10. The process of claim 6, wherein the salt of compound (I) is a monohydrochloride salt, a dihydrochloride salt, a bisulfate salt, a sulfate salt, or a phosphate salt, or a hydrate or solvate thereof.

11. The process of claim 10, wherein the salt of compound (I) is a monohydrochloride salt of formula (XI) 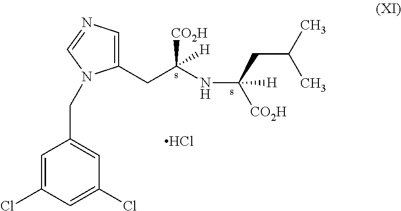
or a hydrate or solvate thereof.

12. The process of claim 10, wherein the salt of compound (I) is a dihydrochloride salt of formula (XII) 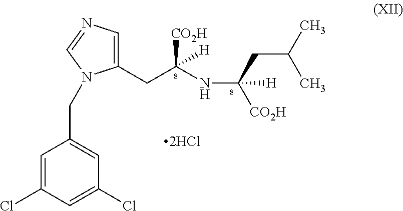
or a hydrate or solvate thereof.

13. The process of claim 9, where the compound of formula (X) was crystallized from an aqueous sodium hydroxide and acetone mixture.

14. A process for preparing Compound (I) 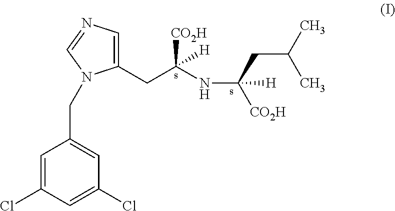
or a pharmaceutically acceptable salt, or hydrate, or solvate thereof, comprising use of an intermediate of formula (VII)

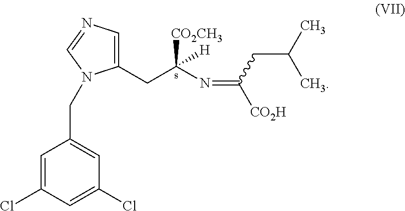

15. A compound of Formula (IX) or a solvate or hydrate thereof

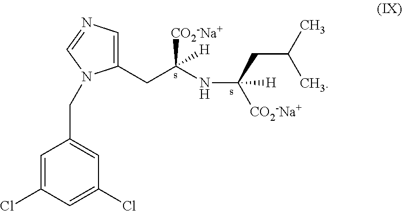

16. The compound of claim 15, which is amorphous.

17. A compound of formula (X) or a solvate of hydrate thereof

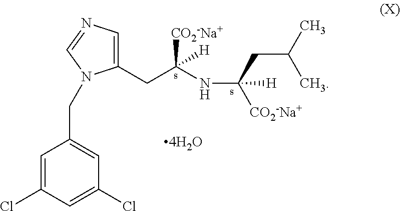

18. The compound of claim 17, which is amorphous.

19. The compound of claim 17, which is crystalline.

20. The compound of claim 19, which is substantially free of amorphous.

21. A compound of formula (XI) or a solvate of hydrate thereof

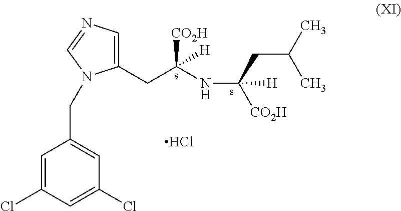

22. A compound of formula (XII) or a solvate of hydrate thereof

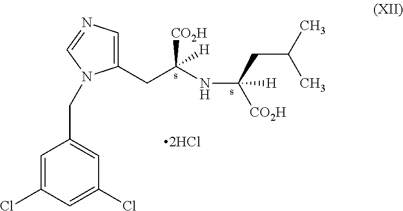

23. The compound of claim 17, as a hydrate comprising up to about 10 mole percent water.

24. A polymorphic form of a compound of Formula (X) 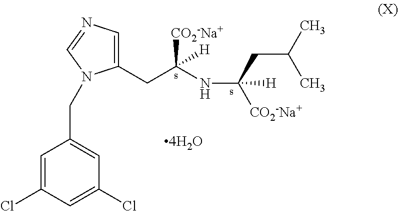
characterized by a powder x-ray diffraction pattern comprising at least one of the following peaks:
2θ
4
16
16.8
19.8

25. A polymorphic form of a compound of Formula (X) 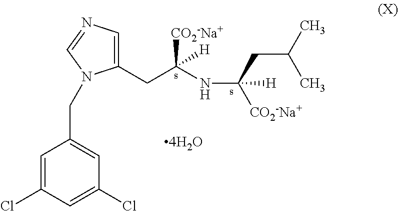
characterized by a powder x-ray diffraction pattern that substantially corresponds to that shown in FIG. 2.

26. A hydrated form of compound (IX) 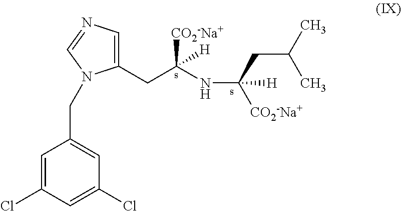
having a sharp endotherm from between about 87 to about 91° C.

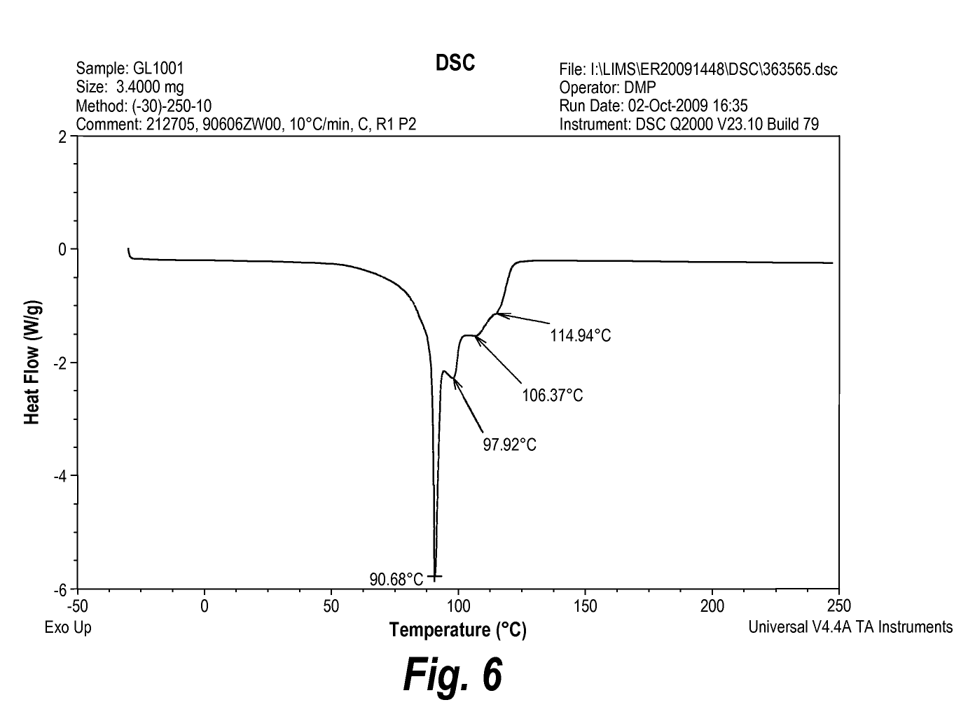

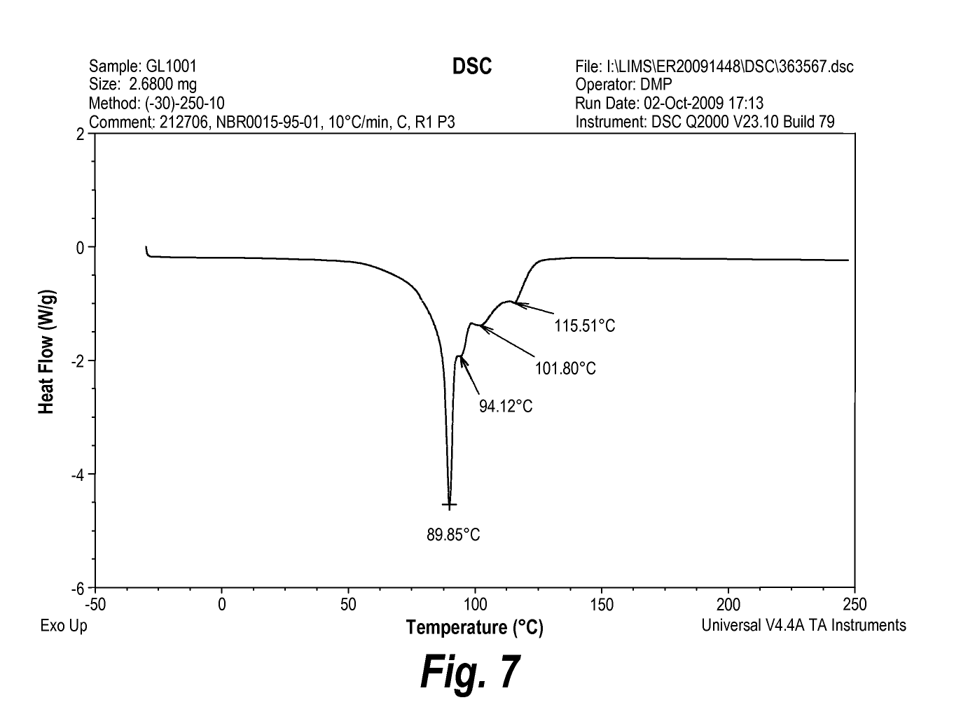

27. A compound of formula (X) 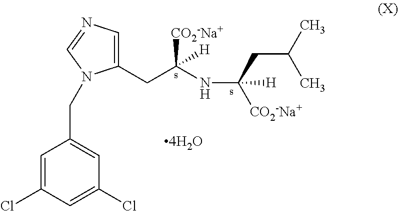
characterized by a differential scanning calorimetry thermogram that substantially corresponds to that shown in any one of FIG. 5, FIG. 6, or FIG. 7.

28. A pharmaceutical composition comprising a compound of claim 17 and one or more pharmaceutically acceptable carriers, diluents, or excipients.

29. The pharmaceutical composition of claim 28 in an oral dosage form.

30. An oral dosage form comprising a compound of claim 17 and one or more pharmaceutically acceptable carriers, diluents, or excipients.

31. A method of treating or preventing inflammatory disorders of the gastrointestinal tract comprising administering a compound of claim 17.
